(19) United States
(12) Patent Application Publication
(10) Pub. No.: US 2003/0108460 A1
(43) Pub. Date: Jun. 12, 2003

(54) Method for surface corona/ozone making, devices utilizing the same and methods for corona and ozone applications

(76) Inventors: Sergey I. Andreev, Richmond Hts, OH (US); Ivan S. Andreev, Richmond Hts, OH (US)

Correspondence Address: SERGEY I. ANDREEV, 667 ALLISON DRIVE, RICHMOND Hts, OH, 44143, US

(21) Appl. No.: 10/314,823

(22) Filed: Dec. 9, 2002

Related U.S. Application Data
(60) Provisional application No. 60/339,190, filed on Dec. 11, 2001.

(51) Int. Cl. B01J 19/08

(52) U.S. Cl. 422/186.07; 422/186.08; 422/186.12

(57) ABSTRACT

A method for making surface corona discharge, which produces ozone gas and apparatus for producing the same are disclosed, in which a dielectric spacer/film having a specific capacity C′ equal to or more than 200 nanofarad per square meter positioned between the base electrode and the net electrode. Said net electrode is a wire net or a perforated metal or a wire winding having an open area not less than about 70%, and a size of hole D equals to or less than about 0.7V/P, where V is a voltage in kilovolts and P is pressure of an ambient air or an oxygen in atmospheres. Net electrode has radius R of wire or radius of an edge of openings in the perforated metal equal to or more than about 1.6d, where d is the thickness of the said dielectric spacer/film, which is determined by a fundamental formula d=9k/C′, where d is in millimeters, C′ is in nF/m², and k is dimensionless dielectric constant k of given material. Under disclosed parameters said surface corona is safe for human contact if said net electrode is grounded. Start voltage Vₛₜ of ozone production is determined by experimental formula Vst=0.7+60/C′, kV, here C′ in nF/m². The even and high intensity corona surface and high ozone output takes place if the operating voltage is more than about 3Vₛₜ. AC power supply is applied to produce ozone gas at “home” voltage 0.7-1.0 kV and produces ozone gas more effectively at 2,5-3.6 kV. A method for disinfection and decontamination of objects by using the direct corona contact and apparatus for making the same is disclosed, in which safe corona surface is placed on said object and is acting by ozone, ultraviolet and ion bombing simultaneously in the unique environment, which takes place inside of the safe corona. A method for ozone disinfection and decontamination and devices for making the same is disclosed, in which said ozone generating element is placed in a closed container/room with or without treated objects and produce a high ozone concentration due to effective ozone dispersion from corona surface without blowing of air/oxygen through said container/room.

This patent is based on the provisional Patent Application No. 60/339,190, filed Dec. 11, 2001.

### REFERENCES CITED

U.S. PATENT DOCUMENTS

| | | | |
|---|---|---|---|
| 4,614,573 | September 1986 | Masuda et at. | 204/176; |
| 4,666,679 | May 1987 | Masuda et al. | 422/186.2 |
| 5,364,600 | November 1994 | Stiehl et al. | 422/186.07 |
| 5,147,678 | Sep. 15, 1992 | Foerch et al. | 427/40 |
| 5,292,479 | Mar. 08, 1994 | Harada et a. | 422/005 |
| 5,429,743 | Feb. 27, 1996 | Schneider et al. | 8/149.2 |
| 5,868,999 | Feb. 09, 1999 | Karison et al. | 422/030 |
| 6086833 | Jun. 11, 2000 | Conners, et al. | 422/292 |
| 4549477 | Oct. 29, 1985 | McCabe, Jr. | 99/477 |
| 6,007,770 | December 1999 | Peiper et al | 422/22 |
| 5,501,844 | Mar. 26, 1996 | Kasting et al. | 422/186.15 |
| 1,796,110 | Mar. 10, 1931 | F. Lechler. | |
| 6,080,531 | Jun. 27, 2000 | Carter et al. | 430/329 |
| 6,115,862 | Sep. 12, 2000 | Cooper et al. | 8/158 |
| 4019986 | April 1977 | Burris et al. | 210/139 |
| 5,430,228 | Jul. 04, 1995 | Ciambrone et al. | 588/200 |

FOREIGN PATENT DOCUMENTS

| | | | |
|---|---|---|---|
| 2,024,427 | Dec. 15, 1994 | Andreev | Russian Patent Off. |
| 08,238,323 | February 1998 | Kozo | Japan Patent Off. |

### BACKGROUND OF INVENTION

1. Field of the Invention

The present invention, in general, relates to a method for making surface corona discharge that produces ozone gas, devices utilizing the same and methods for surface corona and ozone applications in environmental, food, medical, chemical, and electronic industries.

More specifically, the present invention relates to a method for creating corona, which start producing ozone at the voltage less than one kilovolt and can be touch by hand at the corona discharge voltage up to 4 kV. Safe corona surface can work as an active substance to be placed on the subject to decontaminate it of harmful bacteria (including Anthrax), virus, noxious chemicals, and harmful biochemical substances. The subjects can be a human/animal body, letters, documents, banknote, carpet, walls of surgical rooms, etc. Safe corona has some special applications, such as: removing an unnecessary substance from a silicon substrate surface, discharging of electrostatic electricity from papers and textile, creating plasma cathodes for electron/ion accelerators (guns), initiating power gas lasers, etc. The direct corona contact with human/animal body and other subjects is a new method for application, presented in this patent and is referred to as Direct Corona Contact method (DCC).

Safe corona/ozone generating element practically is a self-sufficient device—ozonizer or corona reactor that can work without feeding gas (oxygen or air), but generates ozone from the ambient atmosphere. This fact opens new possibilities for ozone generating element application, in which thin generating ozone plate is placed in a closed container/vessel with a treated objects such as a food, infected material, washing/drying articles, etc. and treats said objects without the blowing of oxygen containing gas through said container/vessel as in existing applications. According to present paten ozone disperses from corona surface and files container/vessel in very short time. Ozone Dispersion (OD) method is convenient for sterilization of medical equipment, hospital rooms, public accommodates, trucks, ship hold, etc.

This patent presents some devices to demonstrate the possibilities of named above three methods: creating safe low voltage corona/ozone generating element, Direct Corona Contact-DCC method and ozone disperse OD methods for applications.

2. Description of the Prior Arts

The most effective method for production of ozone gas is high voltage corona discharge. Various apparatus and methods have been proposed for corona/ozone making, but all of them have several serious flaws. First, they all operate at a very high and therefore dangerous voltage that result in large, bulky and heavy generating apparatus. Second, the flowing of oxygen containing gas (air or oxygen) through the corona discharge and treating camera is required. Gas blowing system stipulates complicate treating equipment and in addition to being costly. Third, ozone output for existing devices is small if it calculated as a ratio of ozone output to ozonizer size or weight.

The authors of this patent made a research with the goal to find such conditions of a corona discharge that lead to improved corona reactor/ozonizer's safety and at the same time have high ozone productivity at a low voltage. If the voltage less than 3.6 kV size and weight of the corona reactor/ozonizer can be decrease significantly according to International Electrical Classification—IEC safety requirements.

The physical research of corona discharge occurring on a dielectric surface that is placed on a metal surface led to discovery of the method for making optimized corona/ozone generating element that has safe corona surface and high ozone output by using 60 Hz voltage less than 3,6 kV. It was found that the lowest voltage to result in electrical discharge in ambient atmosphere is only 700 volts. This result important not only for ozonizer technology, but for high voltage equipment also.

An appearance of short current pulses that follow with high repetition rates (about thousand pulses per second) results in a sharp increasing of the corona intensity and therefore ozone output, this occurs at the discharge voltage equal or more than 3Vₛₜ, when 60 Hz sine voltage is applied. Thus it is possible to have a substantial increase of ozone output at 60 Hz which will be equivalent to using 600 Hz frequency of ozonizer power supply. An electrical turbulent wind appears above and inside of corona, when said current pulses take place. This wind lead to effective circulation of air/oxygen gas in corona and fast ozone dispersion from corona surface without blowing.

A corona discharge can be absolutely safe for the contact with human body at up to about four kilovolts (4 kV). This effect opens new possibility for applying the surface corona as new agent for treatment of any objects (including human/animal body) by high ozone concentration, ultraviolet, and ion bombing simultaneously. Low start voltage, safety and high intensity are new physical effects that are result of a special geometry and materials characteristics and mutual disposition of parts of corona/ozone generating element, and are discovered by present patent as a method for making surface corona discharge producing ozone.

Said corona/ozone-generating element (or simpler “ozone generating element”) comprises a thin dielectric spacer/film that is placed between net and base electrodes, which are connected with AC power supply. Corona discharge is formed in the opening of the net electrode on the surface of said dielectric spacer. The current in corona has normal and tangent components and goes through the dielectric spacer, as through an electrical capacitor. The high voltage capacitor technology is developed for ozone generating element making. The optimal parameters such as a thickness of said dielectric, the size of holes in the net, net open area, the radius of net wire or equal radius of perforation and thickness of net electrode are given. The optimization was found as a compromise between desired parameters and physical constraints.

On one hand the lowest corona start voltage Vₛₜ is desired. It was experimentally found that the Vt depends on the dielectric materials, which usually are used for high voltage pulse capacitor (Mylar, Polyester, Teflon, and some inorganic dielectrics, for example, mica, enamel, etc.) Said material may be characterized by only one parameter—specific capacity C′ if air/oxygen pressure P is given. Many experimental data can be summarized by formula Vₛₜ/P=0.7+60/C′, kV, if C′ in nanofarades per square meter (nF/m²) and P in atmospheres. It is possible to get Vₛₜ less than one kilovolt at one atmosphere as it is shown from this formula, if C′ is more than 200 nF/m². This formula is unique and can also be used in high voltage engineering. The thickness of the dielectric spacer/film can be determined by well-known formula d=9k/C′, millimeters if C′ in nF/m². Here k is the dielectric constant of material that can be chosen from any material catalog. For high C′ said dielectric spacer/film must have a small d.

On the other hand there is a limit for minimal size of d, which is determined by the said dielectric breakdown voltage. Named materials have a large breakdown voltage for small d and thus large specific capacity C′ providing Vₛₜ less than one kilovolt can be realized.

On one hand, it is desired to increase ozone production from unit of generating corona surface. For that, corona in the net holes must form practically even burning surface. It is obvious that net electrode must have a large transparency (or large open area). Wherefore the holes have to be large and wire radius/diameter must be small. On the other hand, the size of the net holes D must be less then D<0.7V/P millimeters, where corona discharge voltage V is in kV and pressure P of ambient air/oxygen is in atmospheres (atm), to fill the holes by corona at given voltage V. This limit of D is determined by experiment. The wire radius R has a low limit, because electrical field strength is inversely proportional to the wire radius R and smaller radius leads to the increase strength in the dielectric and this way increase the probability of the dielectric breakdown. It was determined by experiment that the radius of the net wire has to be more than R>1.6d (the wire diameter must be more than 3.2d), where d is the thickness of said dielectric determined above. The net open area has to be not less than about 70% to form practically even corona surface under disclosed limits of R and D. To increase ozone output and make corona thickness more than thickness h of the said net, the net can be filed as it is shown on the FIG. 1. The filed net has a very small radius of the edges curvature r that lead to an increase in electrical field near said edges making the corona layer thicker.

The intensive corona discharge, which has been made under named above optimized parameters, is safe at up to 4 kV if the said net electrode is grounded. This new phenomenon was discovered by experiment. Safe corona can be used as a new sterilizer acting by ozone, ultraviolet and ion bombing simultaneously on any surface including humans and animals. The Direct Corona Contact—DCC method is presented in this patent. It is extremely effective since corona layer is not only inside the net holes but protrude in front of net surface, especially due to the net having cut edges with a small radius r (FIG. 1).

Ozone optimized generating corona surface can work without feeding the gas (oxygen or air) because corona discharge makes turbulent wind that moves from corona surface. Said wind plays an important role by forcing ambient gases (air or oxygen) into corona for production of ozone and effectively cools the corona and ozone generating element. Said “electrical wind” usually takes place around the wires of high voltage transmission lines and is well documented. Ozone dispersion from corona surface is not only process of diffusion but also is an electrical wind process. As a result ozone disperses much faster than simple diffusion, which was determined by experiment. Therefore Ozone Dispersion (OD) method is offered by present patent as a novel treatment method allowing to obtaining high ozone concentration inside a closed container/room without blowing. The absence of blowing makes ozonizer design and it's applications much simpler and cheaper. According to OD method the ozone concentration in the closed container with ozone generating element inside is determined by the equilibrium between velocity of ozone production and ozone conversion in to oxygen. Ozone concentration can be highest, if the velocity of ozone conversion is much smaller than velocity of ozone production.

Ozone conversion produces oxygen, which is reused for continuous ozone production in a closed container. The common concentration of oxygen in the air inside the container decreases about 10%, if ozone concentration is 48 gm/m³ (or one mole of ozone in the cubic meter) stationary.

Present patent provide optimized parameters of corona/ozone generating element that allow to employ novel DCC and OD methods for corona/ozone applications. Resulting devices can be used for disinfecting infected surfaces, treating polymer surfaces, decontamination of letters, documents, the devices for ozone disinfecting of toilets, articles inside washing/drying machines, medical articles in humid and dry atmosphere, the food and mail articles on a conveyer, cooled articles in soft bag, for facilities deodorization, for disinfecting whole human body, for sterilization of air in ventilator house system, for demolition of oil films on the water surface and removing photoresist, for liquid saturation or food sterilization by ozone in closed container, for ozone treating running film of viscosity liquid and water purification in shower device.

Previous S. Andreev Russian Pat. No2024427, 1994, entitled: “Ozone generator (it's versions)” discloses the surface ozone generator comprising of a dielectric spacer placed between net and metal electrodes. A surface corona discharge is formed in the opening of said net electrode, if AC high voltage is applied between said electrodes. A blowing method was used for surface corona ozonizers. But ozone generating element optimization, provided by present patent, which lead to safe corona and to realization of new DCC and OD methods for application were not discovered by Andreev Russian patent.

U.S. Pat. No. 4,614,573, to Masuda, entitled: “Method for producing ozone gas and apparatus for producing the same”; U.S. Pat. No. 5,364,600, to Steihl et al., entitled: “Apparatus for producing ozone from oxygen” and JAPANIES Pat. No10053402, to Kozo, entitled: “Surface ozone generator element” relates to surface corona ozonizer but does not provide the optimized parameters for surface corona/ozone making provided by present patent.

Next group of the patents are prior art relates to some ozone applications.

U.S. Pat. No. 5,147,678, to Foerch at al, entitled: “Modification of polymer surface by two-step reaction” discloses method of polymer surface modification, in which said polymer is exposed to nitrogen plasma in a primary reaction chamber. Than treated polymer is transferred to the secondary reactor site for secondary treatment by ozone.

The modification of polymer surface can be made without the transfer to another reactor site if disclosing by present patent DCC method is used (see FIG. 5). The corona can be made in the necessary gas for discharge treatment of an object, and corona can be moved to create contact with the object or to distance from the object by increasing or decreasing of a discharge voltage correspondingly. Resulting device is much simpler and treatment process is more convenient. All treating cycles will be shorter because treated object is not moved and productivity of modification process will be higher.

U.S. Pat. No. 5,292,479 to Harada et al., entitled “Air treatment by multi-stacked plate assembly” discloses the method and apparatus for the air treatment, including toilet stool incorporating an ozone deodorizing system equipped with a deodorizer fan.

According to present patent flat ozone generating element is placed on toilet stool cover, having a safe generating surface facing in a bowl (FIG. 7). In this case the sitting is disinfected in direct contact with corona surface and bowl is disinfected and deodorized by OD method without any moving means.

U.S. Pat. No. 5,493,743 to Schneider et al., entitled “Ozone assisted laundry wash process and waste water treatment system” discloses the method and apparatus comprising of wash water system wherein wash water is maintained in a contact tank where it is treated by ozone, which enters the water through the venruti injector. According to present patent flat ozone generating element is placed on the door/lid of existing washing/drying machines (FIG. 8) The grounded net electrode is not affected by spatters of water. Disclosed design uses the OD method and does not require a contact tank or venturi injector. Moving wash water inside the washing machine improves the contact of ozone atmosphere and water, making ozone solution. The attached ozone generating plate on the lid of the drying machine, produce ozone atmosphere in which articles are moving. It is much simpler and cheaper than the system of cited patent.

U.S. Pat. No. 5,868,999 to Karison at al., entitled “Ozone sterilizer and method for ozone sterilization” discloses ozone sterilizer, comprising an ozone generator with feed gas inlet, holding tank is in fluid communication with the ozone generator, a humidification chamber is in fluid communication with the holding tank.

According to present patent ozone generating element is placed in a holding tank with articles to be treated and creates ozone atmosphere inside the tank due to ozone dispersion from corona surface without fluid communication means. The detachable cameras can produce not only humid, but also dry ozone atmosphere too. The option of humid or dry treatment atmosphere depends on specific needs of treatment subjects. After treatment the special lamp destructs ozone (convert in oxygen) in a short time if it is necessary. Thus the device and treatment service is simpler, more universal and cheap (FIG. 9).

U.S. Pat. No. 6,086,833, to Conners et al., entitled “Process and equipment for sanitizing and packaging food using ozone” discloses a device for mixing ozone with a carrier fluid that is used for reducing the microbial content in food. The device comprising ozone generator connects with venturi nozzle, an outlet, two inlets, a blender, etc. Ozonized fluid is injected into container with food product to be sanitized by ozone. Our patent provides a shower method for water drops ozonization and food/objects disinfecting by said water drops and ozone atmosphere simultaneously. Said shower method can be used as a water purifier itself (FIG. 21).

The shower method and apparatus is much simpler and productive than discovered by Conners at al.

U.S. Pat. No. 4,559,477, to Mc Cable, etc., Named “Ozone treatment system for food” discloses apparatus for the treatment of food products, which continuously move on the conveyer through the treatment chamber filled with ozone. A special source of ozone is connected with this chamber to fill it with ozone.

According to our patent ozone generating element is placed inside the container with treated subjects and fills this container by ozone due to ozone dispersion—OD process. Food, mail or other products are conveyed through said container. (FIG. 10). Special means to prevent service personnel from ozone poising are disclosed. To compare our new apparatus with prior art it is necessary to compare common power consumption, ozone concentration in the treatment zone, dimensions and cost of all treating system. Any of named parameters are improved through using our present patent.

U.S. Pat. No. 6,007,770, to Peiper et al., entitled: “Method and a device for ozone sterilization of objects” discloses the method for sterilization of object, including the steps of placing the object that should be sterilized in closed vessel; placing the closed vessel between two high voltage electrodes, etc. Discharge current goes through the said vessel treating articles. Sparks generate ozone and discharge voltage depends on the size of said vessel and electrical parameters of vessel walls. The discharge voltage is high (7-25 kV) and corona discharge is not mentioned in Peiper's patent. Authors mentioned only the sparks, which took place in the described conditions. The bright channel of spark does not produce ozone due to a very high temperature in said channel under what ozone molecule is destroyed. Only corona, which takes place in front of developing sparks produces ozone with very small concentration and minimal efficiency. Moreover with given voltage of 7-25 kV, the size of the said vessel can only be extremely small—about one centimeter, because standard electrical strength for beginning of ionization processes in dry air at one atmosphere is 30 kV/cm.

According to present patent, ozone generating element is placed inside a closed vessel with the object and electrical current goes through inside said generating element and so the work voltage is less 3.6 kV for any size of closed vessel (see FIG. 11). The disclosed device is more convenient for service, more universal can be widely used, for example, for medical articles, foodstuffs, etc.

U.S. Pat. No. 5,501,844 to Kasting at al., entitled “Air treating apparatus and method therefore” discloses the method and apparatus for air deodorizing, comprising a housing with an air inlet and air outlet for air passage through, the blowing means, a 8,000-12,000 volts corona generator that is located in the passageway, etc. According to the present patent low voltage ozone generating element is placed inside deodorized room to make predetermined ozone concentration inside said room without passageway and blowing means. The small, flat generating elements may be positioned on the walls of said room to get even distribution of ozone concentration in the room by using ozone disperse OD method (FIG. 12). The perforated net electrode is grounded and corona is safe. Said net electrode may be executed like a picture, which is seen as a blue glow in the dark. The main advantage of disclosed device is safe corona and safe device without blowing means. This device can be used for hospitals, offices, restrooms, animal containing accommodations, etc.

Corona discharge and ozone using can solve the problem of eliminating harmful gases (such as NOx, SOx, CO, HF, hydrocarbons, etc), that exist in a chimney, car exhaust, etc. U.S. Pat. No. 1,796,110 to Lechler, 1931 “Process and apparatus for effecting chemical reactions between gases” provides a method and apparatus for “promoting chemical reactions by combined action of contact substances and silent electric discharge, in such manner that the electrical discharge acts not only to directly promote the reaction, but also to create electrical wind in the gas so as to bring the gases into more effective contact”. The idea was to use corona discharge, named above as a silent electric discharge, is valuable 70 years later, because any corona produces ozone that shares in chemical reactions as well as plasma-chemical reactions in a corona discharge. This double activator for gaseous reactions are effective and productive, if the appropriate apparatus is created. The apparatus according Lerner patent comprises of a thin wire that is placed along pipe axes and is the corona-making electrode. The gas current goes along pipe axes and perpendicular to said electrical wind. Corona takes place around said wire and produces ozone inside the pipe. A special electrode means cover the pipe inside are used to increase discharge intensity and corona radius because corona cylinder around thin wire has small diameter and limit of work diameter of the said chemical reactor. Of course, the common productivity (cub. meter per hour) of such reactor is limited too. The corona around thin wire that usually takes place in high-voltage transmission lines is thoroughly studied and can be calculated. U.S. Pat. No. 6,334,982 to Carlow et al 2002 named “Corona discharge reactor” provide analogues design using central wire electrode of large diameter (few centimeters) to eliminate the rapid fall-off in the electric field along the radius of the wire. In this case, homogenous corona volume transforms in many streamer channels, which decrease productivity of the reactor due to dark places between said channels. A start voltage for discharge increase with said wire diameter and pulse high voltage is provided to obtain a larger number of said streamer channels. The increase of the cross section of the treating gas passageway in this case does not mean the increase of the reactor productivity. More effective reactor corona was used in U.S. Pat. No. 6,193,934 to Yang, 2001 “Corona-induced chemical scrubber for the control of NOx emissions” providing a corona discharge in the gap between a pair of electrodes having a dielectric barrier between them for inhibiting a streamer/spark discharge. It is a typical ozone generator design in which air containing molecules of NOx is blown through said discharge gap. AC voltage, in this case, depends on the width of said gap and limits the productivity of the reactor. Said voltage usually larger than ten kilovolts.

Present patent offers to use several cylindrical corona/ozone-generating elements placed coaxial to each other to form cylindrical surface corona that generates ozone at 3.0-3.6 kV without dependence from the width of said gaps. The decomposition of harmful gases takes place in the gaps between a few coaxial cylindrical corona surfaces, when said gases flow through this gaps (FIG. 17). The electrical wind from corona surfaces into said gap increase treatment activity Disclosed method provides much more ozone concentration in said gaps (reacting zone) and design has no limit of the diameter because the number of said coaxial cylinders can be increased. Our novel cylindrical shape surface corona design can be conveniently used to improve the combustion process by increasing cetane number and lowering the compression ratio for Diesel engine. The industrial chimney gas can also be purified by using said design.

U.S. Pat. No. 6,080,531, to Carter et al. “Organic removal process” provides a method for removing organic material from substrate by using the water-ozone solution, which according authors opinion “is an attractive method of photoresist removal as it eliminates many of the problems of the traditional photoresist removal methods”. The removal of photoresist from substrates is important for microelectronics; the removal of different organic layers/films is important for optics, technology used to cover tools reinforcing materials (such as drills covered by TiN or CrC) and other industries. A technological scheme according to Carter et al patent comprises of a cylinder that is named by authors as counter-flow contractor that is used for making ozone-water solution. It is a conventional technology, which uses water that flows down and ozone containing in air/oxygen bubbles that go up to increase the contact time and ozone concentration in water. Ozone generator makes ozone-containing air/oxygen gas, which supply a special diffuser for bubble making. Common setting for removal process is complicated and expensive. According to present patent the apparatus for photoresist removal is mush simpler (see description of FIG. 18B). The layer of water is in direct contact with grounded corona (not with net electrode) making a high ozone concentration in said water layer. Said ozone saturated layer that moves slowly along said substrate surface, removing photoresist and other organic films. The organic films on the water (oil film, for example) can be removed according present patent by high ozone concentration in the air gap between corona surface and film on the water surface (FIG. 18A). In this case disclosed ozone generating plate floats on the water. Said apparatus is more effective due to higher ozone concentration, it is simple, more convenient for service (corona surface is grounded and safe), and much cheaper.

U.S. Pat. No. 6,115,862 to Cooper at al “Cold water ozone disinfection” and number of US References provide methods and apparatus for ozone making and ozone-waste water treatment Two methods of ozone-water contact are widely used in preparing ozone solution. One of those methods uses injection means such as a venturi injector and the other uses diffuser means for making ozone containing air/oxygen bubbles in the water. Cooper et al. patent disclose typical apparatus with injector, including ozone generator, ozone containing gas flow means, water flow means, ozone injector, washer chamber with ozonized water, etc. Specific of Cooper's patent is the method and apparatus for re-introducing the ozonized wash liquor.

U.S. Pat. No. 4,019,986 to Burris at al. “Portable water purifier” provides water purifier comprising of an ozone generator, water container, a flexible tube connecting the two, filter that served for bubbling water, etc. Common scheme for the water purification stand providing bubble contact method.

Our patent provides devices, in which a thin liquid layer moves on a substrate (FIG. 18A), or does not move (FIG. 19), or the film of viscose liquid moves (FIG. 20), or a water drops fall (FIG. 21) in ozonated atmosphere. These devices are new because novel OD method is used. According to this method, the contact of ozone gas and the liquid takes place on a thin layer or small drops of said liquid to provide for effective ozone solubility. Such devices are simple and convenient for many applications, including ozone treatment of water, oils, liquid for medicine, wet food, etc.

U.S. Pat. No. 5,430,228 to Ciambrone et al. “Ozone method for the destruction of chemical weapons” provides an ozone neutralization system, comprising of purging means for removing chemical compound from source and neutralizing means for breaking at least one chemical bond in harmful chemical compound. The simple shower is used as the said purging means and bubbling method is used for ozone disinfecting of said removal chemical compound in the liquid. According to our patent (FIG. 21) said system produce ozone atmosphere that ozonized water shower drops, which are used for decontamination and removing of chemical compounds from the disinfecting object simultaneously. Disclosed design is more convenient to use especially in field conditions.

### SUMMARY OF THE INVENTION

The inventors have experimentally discovered that the surface corona intensity and ozone output can be significantly increased at special parameter of the corona/ozone generating element. Low start voltage Vst (less than one kV) and high ozone output at 3Vst, safe corona (can be touch by hand), even corona surface, intensive ozone dispersion from corona surface—all these features are a result of the disclosed generating element creation method that includes the steps:

applying an insulator spacer/film, having specific capacity C′ more than 200 nanofarades per square meter C′>200 nF/m² and thickness d=9k/C′, where k is dimensionless dielectric constant of given insulator material; applying a net electrode made of wire, net or wire winding, or perforated plate having the size D of openings not more then 0.7V/P (D<0.7V/P) where V in kV and pressure of surrounding air/oxygen P in atmospheres, and having the radius R of the net wire or perforation not less than 1.6d (R>1.6d), where d is the thickness of said spacer/film, and having an open area (ratio the sum surface of holes to common surface of the net) not less than about 70%.

applying a base electrode made of metal;

placing said spacer/film between net and base electrodes;

Connecting AC power supply to said electrodes.

According to another feature of present invention, a said ozone generating element can be safely contacted with human body by corona surface up to four kilovolts, if said net electrode is grounded. That is impossible in all the existing ozonizers.

According to another feature of present invention a said ozone generating element can work at the voltage of less than one kilovolt.

According to another feature of present invention, ozone generating element has ozone output about 100 grams per hour of ozone from one square meter at only 3.4 kV, 60 Hz in surrounding humid air and 250 grams per hour in pure oxygen. This voltage is several times less than the existing ozonizers usually use.

According to another feature of the present invention, there is a novel method for corona/ozone applications including the steps of putting said ozone generating element with a safe surface corona on the treated object such as human/animal body in direct contact. That is impossible for the existing ozonizers.

Physically corona is a great number of electron avalanches, developing in a gas under high strength of an electrical field. High-energy electrons produce not only ionization, but they also dissociate the molecules of oxygen forming atomic oxygen, which instantly joins oxygen molecule forming three atomic molecule of ozone. High-energy electrons in said avalanches also excite various kinds of gas molecules. Excited molecule instantly emits quantum of radiation including ultraviolet, which destroys bacteria (this ultraviolet named as germicidal radiation). The most effective part of said radiation has a small free path before being absorbed in air/oxygen and can effective disinfect only the subjects being in-situ of cold corona discharge. When electrical field accelerates electrons, the negative oxygen ions are accelerated too and ion bombing occurs of any subjects that are placed inside corona. So, Direct Contact with corona surface acts by the highest ozone concentration, said germicidal radiation and ion bombing having the strongest disinfecting and decontaminates effect. Direct Corona Contact—DCC method is especially effective for treating human/animal body, surface, letters, banknotes, carpet, soil, room's wall, etc. DCC method solves the ozone-poisoning problem too. Direct contact of the ozone generating surface with a surface to be disinfected does not allow ozone gas to disperse in surrounding air. The volume of ozone containing air in direct contact corona with treating surface is so small that ozone concentration in the environment becomes less than OSHA 8 hour exposition limit 0.1 ppm in surrounding air when power supply is turned off and said ozone generating surface is removed from treated surface. The treatment takes only a short period of time due to high effectiveness of corona action. Ozone molecule is small and can penetrate through the pours of usual packaging materials (including mail's envelopes). Ozone molecule has a size 0.22 nanometer that is less than size of typical paper porous hundred or even thousand times. The effective ozone penetration through said porous requires the difference of pressure, which takes place due to said turbulent diffusion of air containing ozone. Authors of present invention made experiment with perfumed paper, placed in an envelope. It takes only two minute to eliminate the odor from said perfumed paper inside closed envelope.

Direct corona contact (DCC) method allows to effectively destroy of harmful bacteria (including Anthrax), virus, noxious chemical, etc.

According to another feature of the present invention, there is a novel method for corona/ozone applications including the steps of putting said ozone generating element into closed container/room to establish high ozone concentration in said close container/room in a short time due to electrical wind, heat convection and ozone diffusion. Existing ozonizers have corona discharge in a narrow gap between electrodes and thus can not use our Ozone Dispersion (OD) method, since they require oxygen-containing gas to be constantly supplied to the said gap during ozone production. Furthermore, the said invention provides devices for corona/ozone applications that employ above-mentioned DCC and OD methods.

Devices for DCC application method in common comprise of a surface ozone generating element, two electrical insulated conductors that connect net and base electrodes of said generating element with power supply. The said net electrode and the power supply end are grounded. A periphery separator strip/spacer makes a narrow (about one millimeter) gap between net electrode surface and treated subject surface. Each new device has particularities, which depend on the specifics of application.

A design in which it is possible to make corona in different gases for corona treating of a polymer surface.

A design in which two ozone-generating elements are placed face to face to form the gap for both sides decontamination of flat object like letters, banknotes, etc.

A design in which ozone generating element is placed on the toilet cover and disinfect toilet seat, when said cover is down, contacting the seat by corona surface.

A design in which a thin layer of water on the substrate, having the ground potential, is in direct corona contact.

The devices for said OD application method for treating process in closed container/room in common comprise: a closed container/room; said surface ozone generating element inside said net electrode; a treated objects inside; a power supply connected with said ozone generating element and having or not one of the connectors be grounded.

A design in which said ozone generating element is placed on the lid of conventional washing/drying machine.

A design in which medical or food articles can be treated in humid or dry atmosphere as necessary.

A design in which disinfecting food, mail, and other articles are placed on the moving conveyer, which goes through the treatment container that is filled by appropriate ozone concentration and the means to prevent ozone from escaping in the environment.

A design in which medical, garden, food, etc. articles can be sterilized and stored cooled.

A design for room/office deodorization in which said net electrode made of a perforated picture.

A design in which whole human body can be disinfected.

A design in which two-sided ozone generating plate is grounded and safe from both sides.

A design in which said ozone generating element is a metal cylinder, used as a base electrode, is covered by dielectric film, which is tightly pressed to the said cylinder by winded wire that is a net electrode. The water can go through or be kept inside the cylinder if it is grounded for cooling.

A design in which said ozone generating element forms oval with two flat generating sides that is grounded and safe. Said metal oval is insulated inside and can be filled by running water for cooling.

A design in which flowing air of ventilator system or chimney, or exhaust gases are in chemical reaction with ozone. The cylindrical ozone generating elements placed coaxial to each other are used. They have a different diameter and are placed coaxial to each other to form between them the gaps, which are filled by ozone. The harmful gas flows along said gaps where it reacted with ozone.

A design in which plane ozone generating element floats on the water surface forming a small gap between oil film on the water surface and the grounded net electrode to eliminate said oil film.

A design in which any liquid can be saturated by ozone in a vessel having a flat ozone generating element on its lid and treated liquid on the bottom.

A design in which viscous liquid is treated by ozone while a thin film of said liquid goes through a container filled by ozone.

A design in which water is delivered through shower-type device through a high concentration ozone atmosphere. Thus ozone dissolves in small water drops, which can be used to treat various objects.

Novel devices disclosed above have to be optimized according to the subjects of treatment, treatment conditions (temperature, humidity, etc.), and expected type of harmful bacteria/virus or noxious chemical substances that are being eliminated. The most universal device has to be able to make high ozone concentration, which is enough to destroying the most resistant bacteria/virus or noxious chemical substances or cleaning water. For sterilization purpose it is necessary to estimate the highest ozone concentration that is enough for elimination the most resistant bacteria/virus. One of the most ozone resistant microorganism is Staphilococcus albus, that can be destroyed by ozone concentration 2 mg per liter under exposition of 1 minute and Salmonola typhi—destroyed by 2.2 mg per liter and 1 minute exposition. So, ozone generating element has to create ozone concentration about 3 mg per liter and 1 minute exposition in the treating container/room to be enough to destroy any microorganism. Approximately the same ozone concentration is needed to destroy the same microorganisms in water. But in this case ozone concentration in water is 3 to 5 time less than in air and the device producing ozone in air has to be able to produce ozone concentration three-five times more to eliminate the same Salmonola typhi in water. Provided by present patent ozone generating element can create ozone concentration about 100 mg per liter (or 10 g per cubical meter) in air atmosphere and two times more in oxygen atmosphere.

Ozone can be dangerous at long exposures and high concentrations for humans. Special means have to be used for its fast conversion in to oxygen before treat container/room can be safely opened. The devices that use Ozone Dispersion (OD) method, mentioned above, can use UV radiation for closed volumes after treatment (FIG. 9) or flow ozone containing gas through the system of narrow channels (FIG. 10), or use special granules (FIG. 17) to convert ozone in oxygen.

### BRIEF DESCRIPTION OF THE DRAWINGS

The drawings may not necessarily be to scale and features of the present invention may be shown in somewhat schematic form. Nevertheless the various advantages of the present invention will become apparent through the following specification and by reference to the drawings in which:

FIG. 1 is a cross section view of the corona/ozone-generating element with the claimed sizes and streamline of electrical field/current.

FIG. 2 is a graph of the experimental data of ozone specific output (g/h from every m²) as a function of the 60 Hz corona discharge voltage.

FIG. 3 is a comparison of ozone specific output data according to present patent and a conventional tubular glass ozonizer that is supplied by 60 Hz and 600 Hz voltages.

FIG. 4 is a graph of the experimental data of ozone concentration (mg/L) near corona surface that depends on velocity of airflow (L/h) along said surface.

FIG. 5 is a cross-section view of the device for disinfecting/decontaminating of infected surface or treatment of polymer by safe surface corona by using the Direct Corona Contact (DCC) method.

FIG. 6 is a cross-section view of the device for disinfecting/decontaminating of both sides of letters, papers, textile, etc. by-using the Direct Corona Contact.(DCC) method.

FIG. 7 is a perspective view of toilet disinfecting/deodorizing device that uses the Direct Corona Contact (DCC) and the ozone dispersion (OD) methods.

FIG. 8 is a perspective view the washing/drying machine for ozone disinfection by using ozone dispersion (OD) method.

FIG. 9 is a cross-section view of the device that uses ozone disperses (OD) method for sterilization of any objects in humid or dry atmosphere.

FIG. 10 is cross-section view of the disinfecting stand with a conveyer for treatment of objects by using ozone dispersion (OD) method.

FIG. 11 is a perspective view of the device for ozone sterilization and storage of objects in soft plastic bag by using ozone dispersion (OD) method.

FIG. 12 is a perspective view of the ozone-generating plate for air deodorization by safe (grounded) corona that is formed in the slots of perforated net electrode, having a perforation in a shape of picture.

FIG. 13 is a cross-section view of an ozone—chamber for disinfecting the whole human body by using ozone dispersion (OD) method.

FIG. 14 is a cross-section view of two-sided, safe and flat ozone generating device for ozone dispersion (OD) applications.

FIG. 15 is a cross-section view of the cylindrical ozone-generating device with safe corona.

FIG. 16 is a cross-section view of two-sided, safe, oval shaped ozonizer with cooling by running water.

FIG. 17 is a cross-section view of the air-ozone purifier for ventilation, car intake air systems and like.

FIG. 18 is a cross-section view of the device for ozone demolition of oil films on the water surface (A) and for removing photoresist and other organic films from substrate (B).

FIG. 19 is a cross-section view of the device for liquid saturation by ozone including wet subjects in ozone atmosphere by using ozone dispersion (OD) method.

FIG. 20 is a cross-section view of the device for ozone treatment of viscous liquid film by using ozone dispersion (OD) method.

FIG. 21 is cross-sections view of the device for ozone treatment of the drops of water in shower and for object sterilization of by ozonized shower.

### DETAILED DESCRIPTION OF THE PREFERED EMBODIMENTS

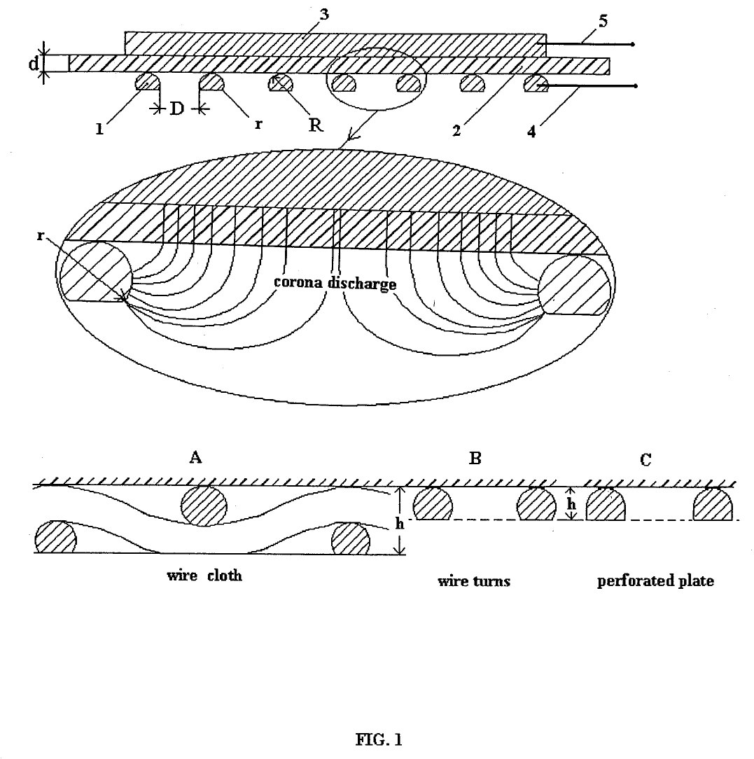

FIG. 1 is a cross section view of the corona/ozone-generating element with the claimed sizes. The streamlines of the electrical field or corona current are shown. Corona discharge has a larger intensity where said streamlines have larger density. When the sine voltage rises, corona discharge disperses inside of holes D in the net electrode 1 on a dielectric spacer/film 2. Said dielectric is placed between said net electrode 1 and rigid base electrode 3. AC voltage is applied by connectors 4 and 5 to the net electrode 1 and to the base electrode 3 correspondingly. The high voltage capacitor technology is used for creating said ozone-generating element. According to this technology it is necessary to have a tight contact between said dielectric spacer/film 2 and the base electrode 3 and said dielectric spacer/film must have a higher capacity and high enough breakdown voltage at the same time. The corona discharge is located in series with said dielectric capacity and the capacity reactance limits corona current. Larger current takes place for larger dielectric capacity. A specific capacity C′ of said dielectric spacer/film depends on the dielectric material thickness d and dielectric constant k according to definition of specific capacity

C′=9k/d,

where C′ in nanofarades per square meter (nF/m²) and d is in millimeters. High specific capacity allows to increase not only corona current and intensity, but to decrease the start corona voltage Vₛₜ too. It was found experimentally that Vₛₜ can be determined by simple formula

Vₛₜ/P=0.7+60/C′, kV,

if C′ in nF/m² and P in atmospheres. It was also found that corona intensity and ozone output depends only on the value of the start voltage if air/oxygen pressure P is given and it is necessary to apply voltage of more than 3 Vₛₜ to get large ozone output. This formula was establish for thin dielectric materials (less than 1 mm), which are usually used for high voltage pulse capacitor such as Mylar, Polyester, Teflon, etc and some inorganic dielectrics, such as mica, enamel, etc. According to this formula the value of Vₛₜ is less than one kilovolt at one atmosphere can be realized, if C′ is more than 200 nF/m². This is a new result, which is important not only for ozone technology but also for high voltage technology in common. The thickness of the dielectric spacer/film d can be determined from formula d=9k/C′, which is equal to given above formula for C′. If C′>200 nF/m² the thickness must be less than 0.045k, mm. The dielectric constant of given material k can be taken from any material catalog. For example, Maylar has k=3.2 and thus d must be less than 0.14 mm if it is necessary to get a Vst<1 kV at one atmosphere pressure. Thinner d leads to more chances of breakdown of said dielectric. Named materials have large enough breakdown voltage to insure a long work life without dielectric breakdown (for example, Maylar breakdown voltage for d=0.14 mm is about 28 kV).

Corona discharge is formed in the opening of the said net electrode on the dielectric spacer/film. The cut net electrode has a very small radius r of cut edges that it is shown on FIG. 1. The electrical field and corona intensity increases near said edges and corona thickness becomes more than thickness of the net electrode. This effect is important for direct corona contact—DCC method, which can be used for many applications. The burning corona can be safe, if net electrode is grounded and net holes size D (for round hole D is a diameter, for slit D is a slit width) is small enough to fill the hole by corona at work voltage. Experiment shows that hole is filled by corona in the maximum of sine voltage, if D is less or equal to

D<0.7V/P, mm,

if V is in kilovolts and surrounding pressure P of air or oxygen is in the atmospheres. The corona is safe and can be touch by hand, if the net electrode is grounded, the corona discharge voltage is not more than 4 kV and size D do not overpass the determined above limit. This invention allows to use 60 Hz power supply with voltage low 3.6 kV voltage to create said corona/ozone generating element under the lowest high voltage requirements of the International Electrical Specification—IES. When the voltage is less than 3.6 kV, surface corona produces ozone with minimal concentration of undesirable nitrogen oxides NOₓ. To increase ozone production from unit of generating corona surface the net electrode must have a large transparency (open area). Said transparency has to be not less than about 70% (shadow surface less than about 30%). Said transparency is enough to form practically even corona surface. To create high net transparency it is necessary to have the hole size D larger and net wire radius R smaller. But value of D is limited as pointed out above and should be not more than 2.5 mm at 1 atmosphere pressure. The radius R can not be too small because the strength of electrical field near any wire under voltage V, is E=V/R and E increases if R decreases. If a net electrode is placed on the dielectric surface the value of E is the strength of electrical field in net-dielectric contact point/line thus increasing E lead to increase in the dielectric breakdown probability and to decrease in the ozonizer work life. If the net electrode is placed above said dielectric spacer/film forming a small gap, variable voltage leads to the vibration of the net and thus frequent hits of said dielectric by the net, decreasing its lifetime. A large gap would lead to a high work voltage. It was determined by experiment that the radius of the net wire must be more than 1.6d (R>1.6d) and the wire diameter must be more than 3.2d. However, the side that has been cut, which is not in contact with said dielectric has a large electrical field E=V/r near cut edges, increasing corona thickness. The cut net electrode on its open side makes the edges with a very small radius r (FIG. 1). The field E near radius r is larger than near net-dielectric contact because R>r. Due to small r, the thickness of corona becomes more than the thickness of net electrode. At the same time the grounded and cut net electrode is safe and can be touched by hand at last up to 4 kV.

The ozone-generating element, which satisfies to all named optimizations, is new.

FIG. 2 shows experimental data of ozone specific output (ozone production in grams per hour—g/h from every square meter—m2) G,g/hm2 as a function of the 60 Hz corona discharge voltage V, kV. A start voltage is lower than 1 kV and a sharp increase of ozone specific output takes place after critical voltage V_{c}, which is approximately equal to three start voltage V₀=3Vₛₜ. Said increase of output is about 70 g/hm² per every kilovolt over critical volta m² ge V_{c} at one atmosphere air. The specific output is G=100 g/h from m² (9,3 g/h from ft²) at 3.4 kV according to experiment that is shown on FIG. 2. In oxygen atmosphere ozone output is two times more. Thus in oxygen, G is about 200 g/hm² (18,6 g/hft²). The output is larger, if said base electrode is cooled. The power consumption is as small as about 15W and about 7W to produce one gram of ozone per hour from air and from oxygen correspondingly. This is corresponding to the specific energy consumption about 15 W per corona surface 100 cm² or 140 W per ft². The consumption can be less if base electrode is cooled. In this case corona is cooled too though a thin (about 0.1 mm) dielectric spacer/film that is in tight contact with said base electrode.

It is well known that molecular nitrogen in air does not react with ozone at room temperature and fast reaction forming nitrous oxide N₂O takes place at 295-300° C. Small specific heating is about 0.15W/cm2 and effective heat transfer allows to eliminate or substantially decrease production of nitrogen oxides by ozone generating element described in present patent.

The specific weight of ozone generating plate in the experiment that is shown on the FIG. 2 was about 2 kg/m² that correspond to the specific ozone productivity about 1 g/h per 20 g of weight. According to present patent, ozone-generating element, having all sides safely grounded, can be used as a self-sufficient safe ozone-generating device with open ozone generating safe corona. In this case ozone productivity and output from unit of weight of the discovered ozonizer is about hundreds times more than conventional ozonizers have.

It is known that ozone output decreases with humidity, thus dried air is used, making ozone production costlier. In our ozonizer the decrease in ozone output in air having 100% humidity can be completely negated by increasing operating voltage by only 0.5-0.7 kV, eliminating the need for air drying equipment, resulting in more cost effective ozone production and a lighter and simpler unit.

Conventional ozone production takes place when air or oxygen is blown through the corona discharge in the gap between paired electrodes. In this case air/oxygen blowing is necessary for generating ozone. But present patent discloses ozonizer, with an open surface corona, that disperses ozone effectively without blowing due to electrical wind and diffusion.

All discovered features result in reduced weight, dimensions and costs, makes the designs simpler, safer and more convenient for operation.

FIG. 3 shows the comparison of the ozone output from the ozonizer, which is designed according to the present patent and conventional ozonizer with tubular electrodes that has air gap between them and glass dielectric that covers one of the said electrodes It is shown that ozone-generating element according to present patent has the same slope of ozone output at 60 Hz voltage that a conventional ozonizer has it at 600 Hz. Special devices for multiplication of voltage frequency make ozonizer and it service more complicated especially for high power ozonizers. The corona current oscillogram explains this effect by an appearance of high repetition rates current pulses at 60 Hz supply voltage. These pulses appear when applied sine voltage decreases from maximum and can be explained as particular discharges of said spacer/film capacity through the corona resistance.

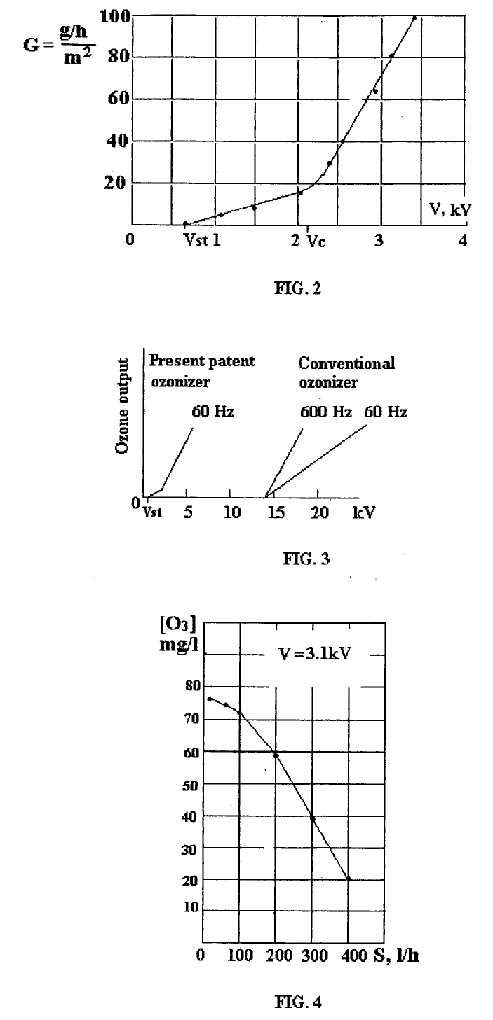

FIG. 4 shows that the higher ozone concentration [O₃] is about 75 mg of ozone per liter takes place, when work voltage is 3. 1 kV and a velocity of the flowing air S is less than 50 liters per hour. Corresponding experiment was made by using two discovered by present patent flat ozone generating elements, which were placed one under another, forming two millimeter gap between corona surfaces facing each other. The ambient air was blown through this gap. The extrapolation of this data to oxygen atmosphere and well-cooled base electrode of the ozone generating element, shows that ozone concentration can be about 200 mg per liter near the corona surface.

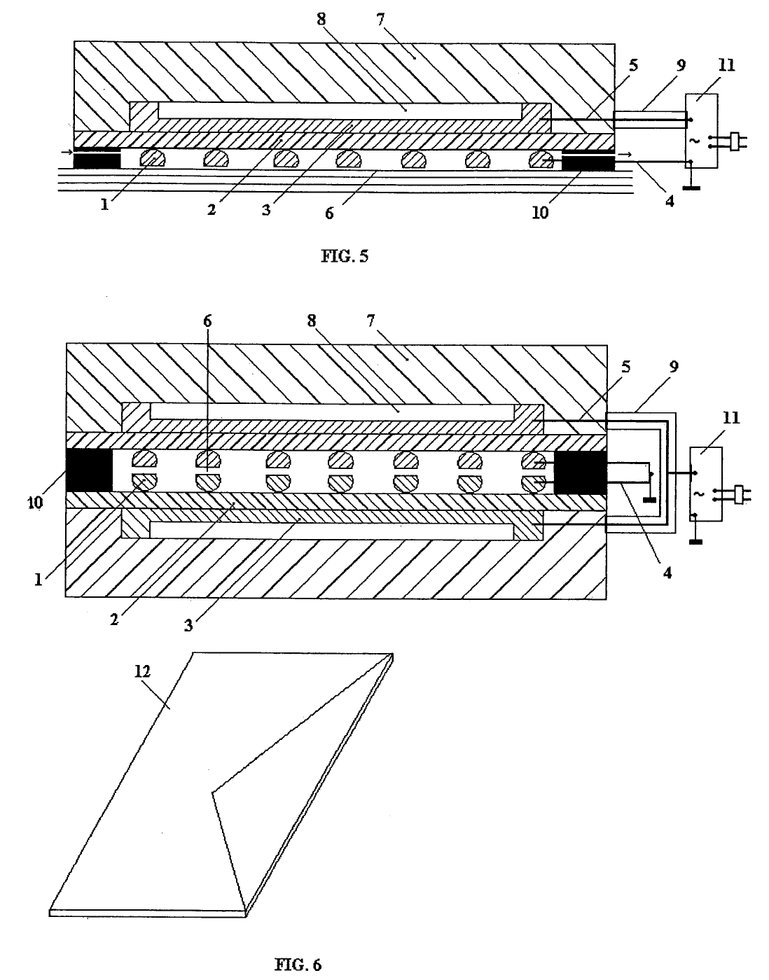

FIGS. 5, 6, and partly 7 show the devices for using Direct Corona Contact (DCC) method in various applications. Said DCC method has unusual high effectiveness due to the fact that it acts by super high concentration of ozone, bactericidal ultraviolet and ions bombing simultaneously in the unique environment, which is the corona. DCC method solves the ozone-poisoning problem. Direct and tight contact between corona surface and a surface to be disinfected does not allow ozone gas to disperse from corona in the environment, or make said dispersion so small that the ozone poison defense can be done by simply using a regular fan. The corona treatment takes only a short period of time due to high effectiveness of the DCC method.

Ozone molecule is small and form isosceles triangle having an oxygen atom on the every apex of said triangle. The biggest is a triangular base, having the length 0.22 nm. Such small dimensions are about hundreds times less than porous size the common paper that allows to ozone penetrate through packaging materials including envelopes.

FIG. 5 is a cross-section view of the device for disinfecting/decontaminating infected surface or treating of polymer surface by safe surface corona in different type of gases (Nitrogen, for example) by using DCC method. The corona surface takes place in the holes of the said net electrode 1 and protrudes outward so that a treating surface 6 is in contact with corona. The flat surface of the cut net electrode stipulates sharp edges of wire, having a very small radius r (see FIG. 1). Near these sharp edges the electrical field is stronger and corona protrudes in front of the surface of the net and contacts the surface 6 that is to be treated. The direct and safe contact with corona has been tested experimentally. The dielectric spacer/film 2 and the dielectric lid 7 form an insulated covering of the high voltage base electrode 3, which is connected to a power supply by wire 5 and cable 9. Conductor 4 dependably grounds the net electrode 1. If dielectric 2 break down, the current between electrodes increases sharply and will be switched off by the relay or fuse (not shown). The lid 7 has a slot 8 for cooling the electrode 3 by air or water. The spacer 10 is placed around said net electrode to prevent ozone dispersion in the ambient air and to regulate the gap between said net electrode and treating surface. It is possible to create corona in different gases, for example, in nitrogen, as Foerch at al. discovers in the US patent (see above) for modification of polymer surface. The corona feeding gas can be substituted through a series of small holes in the said spacer. The arrows on FIG. 5 show a flow of the gas along net electrode surface through said holes. After the gas fills said gap, the holes in the said spacer are closed. For common corona applications, when ozone is used as a part of corona treatment agent, said holes in spacer 10 are not necessary.

After the treatment the ozonizer is switched off and the ozone gas is converted into oxygen in a short time due to a small volume of said gap filled by ozone containing gas. About one minute after ozonizer is switched off, ozone concentration drops and the device can be opened.

This design can be used for any object surface treatment, including human/animal body, papers, textile, carpets, room's walls, soil polymer, silicone wafers, etc.

FIG. 6 shows the device for desinfection/decontamination the letters, paper, textile, etc from both sides. Object 12 as a letter, for example, is placed in the gap 6 between two net electrodes 1 tightly. This design can work like a press to press the object between two corona surfaces for a short treatment exposure. Spacers 10 on the periphery are used to prevent ozone penetration in the ambient air and to establish a gap between net electrodes. Corona is safety grounded by connectors 4. The base electrode 3 connected with high voltage end of the power supply by wire connector 5 and cable 9. Electrode 3 can be cooled by using slot 8 in the lid 7. Dielectric lids 7 make the device operation completely safe form high voltage damage and ozone odor. The conventional receiver form banknote-acceptor machines can be used to automatically feed objects to be treated.

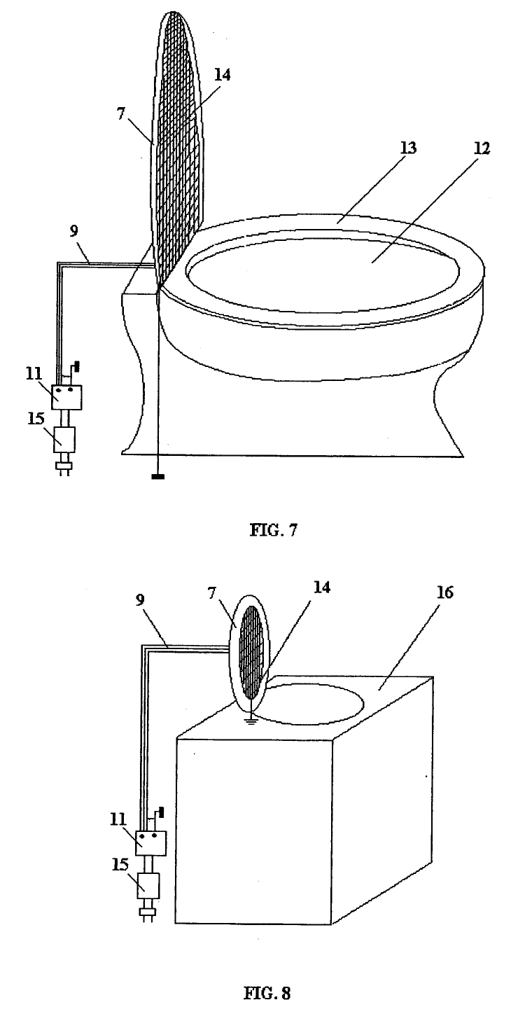

FIG. 7 shows the device for disinfecting and deodorizing a toilet by Ozone Dispersion OD and Direct Corona Contact DCC methods. Flat ozone generating element 14 is placed on the inner surface of a seat cover 7 facing a toilet bowl 12. In the ‘closed’ position corona is in a direct contact with a seat 13 disinfecting it effectively The bowl 12 is disinfected by ozone gas that fills it. The net electrode of said generating plate 14 is grounded and safe at all times. The base electrode (not shown) is insulated by toilet cover 7 and safely connects high voltage electrode by cable 9 with a power supply 11. The timer-relay 15 turns off the power supply automatically after a fixed period of time. The ozone-generating element turned on, when the seat cover 7 covers seat 13.

FIG. 8 depicts a simple application of the OD method for ozone disinfection/sterilization of various objects in washing/drying machines 16. The generating element 14 is fitted tightly to the lid 7 and has a grounded corona surface that is facing inside the machine, where the objects are placed. The HV base electrode is insulated by dielectric of said lid and connected by cable 9 with power supply 11. Said ozone generating element switched on at the moment, when said lid is shut down. A relay-timer 15 is used for switching off the power supply after reaching necessary exposition that is necessary for ozone treatment. Practically it takes only several minutes for ozone disinfection process to be effective. After that interval of time ozone generating element is turned off and the washing/drying machine continue work in the ozone atmosphere. During this time, ozone-oxygen self-conversion takes place and the machine can be opened. In a humid atmosphere said conversion takes about ten minutes. After drying process in ozone containing gas the clothes have a specific fresh smell. Ozone concentration in the cool water is much higher than in a hot water. So, treatment effectiveness may be higher, if washing process takes place in cool ozonized water. The sterilization in cool water is important for articles that can not be boiled.

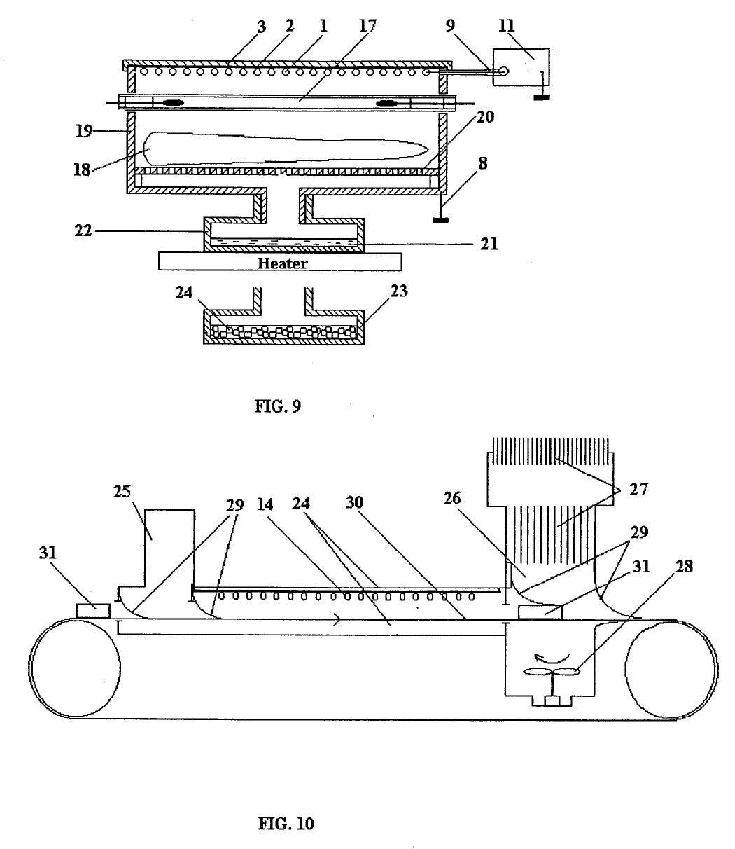

FIG. 9 shows the device for disinfecting different objects 18 inside a closed metal room/vessel 19. Grounded metal lid is used as a base electrode 3 of said ozone generating element. A high voltage net electrode 1 connects by insulated connector 9 the power supply 11. The OD method creates high ozone concentration inside of said room/vessel. If the disinfection must be made in a humid atmosphere, a heater is used to rise the temperature of the water 21 in order to intensify the evaporation process. The water is poured into a detachable metal tank 22. If the disinfection must be made in a dry atmosphere the detachable dryer 23 is used. Silica gel 24 in the dryer 23 is used. A flash lamp 17 (for example, Xenon lamp) is used to convert ozone into oxygen in a short time. Depicted design can have different sizes and different shapes. In a humid atmosphere, ozone conversion into oxygen takes a very short time and the use said lamp is often not necessary.

This device can be used for medical instruments, medical soft objects, food, etc. Currently existing systems comprise an ozone generator with feed gas inlet, a holding tank, which is in fluid communication with ozone generator, a humidification chamber, which is in fluid communication with the holding tank, etc. are much more complex, expensive and less convenient to use and service.

FIG. 10 depicts ozone conveyer stand for disinfecting food, mail products, clothes, instruments or other objects 31 that are placed on the conveyer line 30. Flat ozonizer 14 is placed inside the quasi-closed room 24, with corona surface facing the conveyer line. The room 24 serves the purpose of localizing high concentration ozone inside by using said ozone dispersion OD method. Flexible screens 29 together with the entrance 25 and exit buffer 26 volumes prevent the ozone from escaping the closed room 24 into the surround air. Ozone many-sectional converter 27 is placed in the exit buffer volume 26 to accelerate ozone conversion to oxygen on the large surface of numeral gaps that are formed by plates 27. The fan 28 blows an ozone-containing air across the direction of the moving conveyer line forming a gas shutter. The human nose can sense ozone concentrations of less than permissible dose. Slight ozone smell can be found after the natural lightning. Such ozone concentration (about 0.1 ppm) is safe and beneficial for human health.

The advantage of disclosed design is the absence any means outside, such as ozone generator, adapters, junction means, etc. As a result ozone conveyer stand, made according to present patent, is simpler, cheaper and more convenient for use.

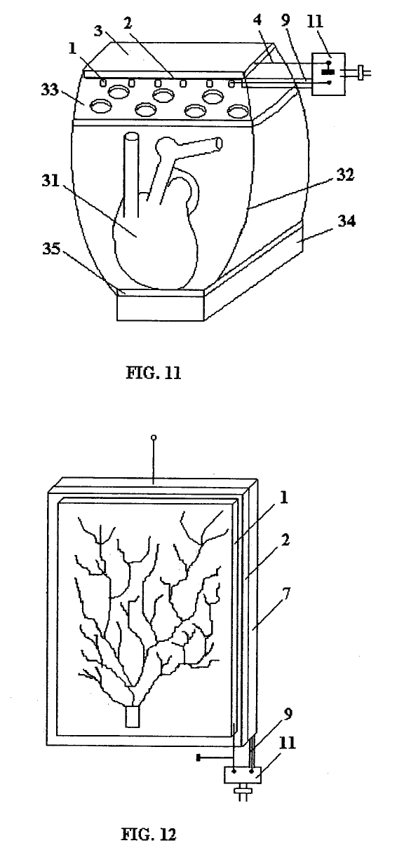

FIG. 11 shows the device for ozone sterilization of objects in plastic bag by using ozone dispersion (OD) method and keeping said objects in cold. Such objects can be tissues, organs, various medical supplies, food products and other things that must be kept sterile for a long time. Said object is placed inside a plastic bag 32. A base metal electrode 3 is a rigid lid of said bag and is grounded. A generating surface corona net electrode 1 faces the treated object 31 and is separated from said object by a dielectric plate 33 having numerous openings. Ozone disperses from corona surface according to said OD method. The net electrode is connected with high voltage end of power supply 11 with insulated wire 9. Box 34 is used to keep the treated object refrigerated or dry, if it is necessary. Said box is made of heat insulated material. Box's lid 35 is a bottom of the bag. The object can be stored in said plastic bag and can be ozonized periodically. Poisoned object can be treated inside said bag and throw away for burial together with an inexpensive ozone generating plate that is disconnected from the power supply 11 and attached box 34 depicted ozone device is the simplest to maintain and operate and very cost effective.

FIG. 12 is a perspective view of the ozone-generating plate for air deodorization by safe (grounded) corona that is generated in the openings of the net electrode, for example in the slots of perforated metal. The perforation can be made as a picture, which is blue in a dark.

Said net electrode 1 is grounded and safe. Said dielectric spacer/film 2 is placed tightly on the base electrode, which is covered by insulator 7 and connected with high voltage end of power supply 11 by insulated wire 9. The treating of houses, public rooms, hospitals, offices, hotels, etc. can use two or more described ozone generating plates to make an even distribution of ozone concentration by ozone dispersing from corona surface. The main advantage of disclosed device is in safe corona, absence of blowing means, picture view, the lowest operating voltage (about 3 kV) simple and cost effective design.

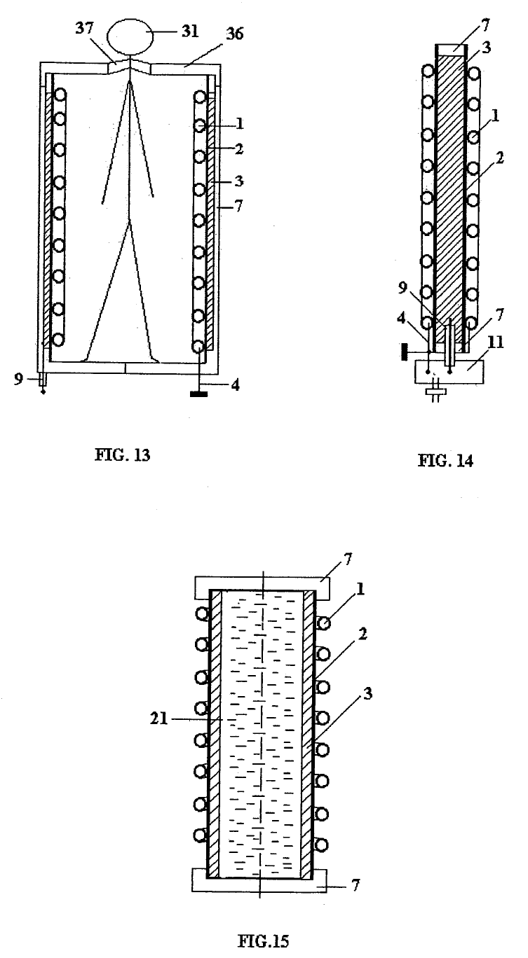

FIG. 13 depicts the device for ozone disinfection of the whole human/animal body by using OD method. A patient 31 is placed inside box 7 with a lid 36. Grounded net electrode 1 generates even corona surface if high voltage more than 3 Vₛₜ is applied. The dielectric spacer/film 2 is placed tightly to said base electrode 3 that is covered outside by insulator 7 and connected with power supply by insulated wire 9. An elastic material like a “scarf” is placed in the hole 37 to prevent a patient from ozone poisoning. The floor of said box is made of an insulator. Depicting ozone chamber is filled even by ozone due to ozone dispersion from corona surface. The ozone-generating element is turned on only after a patient is placed inside. The voltage grows gradually. The design is completely safe and in the case if dielectric 2 is broken down the current arises sharply and fuse cut an electrical circuit, but grounded net electrode keep zero potential at all times. Said net electrode is not made as a winded coil to avoid the voltage arising on the coil when current rises sharply. The net must be made of mesh or perforated metal sheet. This design has no prior art, because save corona surface is disclosed for the first time by this patent. Depicted design very useful, if wounded/burned body must be disinfected.

FIG. 14 shows a cross-section view of a flat ozone-generating device, safe from all the sides, comprising two said net electrodes 1 placed on the both sides of said base electrode 3 and insulated from this electrode by dielectric spacers 2. Electrode 3 is insulated around perimeter by strip 7, which is placed between said spacers 2 as it is shown on the FIG. 14. Insulated connector 9 connects said base electrode 3 with high voltage end of the power supply 11. The net electrodes are connected between them and grounded. So, said ozonizer has all of its sides safe for direct contact and thus is very convenient for many applications. This ozone-generating device is heated by corona discharge and after some time the temperature become constant due to losses of heat by radiation, air convection movement and turbulent electrical wind near corona. All these mechanisms of heat loss arise when the temperature goes up. Provided surface corona has small enough heating power per surface unit, which is about 0.15 Waft per one cm2 (or about 1 Watt per square inch) for every ozone generating surface. In this device corona surface discharge is a heater having heat power about 1W/in2 and at the same time it is a cooler, which determines a low enough stationer working temperature. Disclosed two-side safe (grounded) plane ozone generating device—ozonizer is designed for making ozone atmosphere in closed room/container/due to ozone dispersion—OD method. The safe ozone-generating corona can be placed in the said room/container, if said room/container are office, basement, ship hold etc.

This ozone-generating device has no direct prior art, because save corona surface is disclosed for the first time in this patent.

For a correct comparison of discovered ozonizer with any existing ozonizer it is necessary to compare all parameters including setting, assembly, treating expenses, etc. Disclosed ozonizer is much lighter and can be set up in any place or room by hand having ozone productivity 100-200 g/h and does not have a blowing/conjunction means and is very cost efficient.

FIG. 15 shows a cross-section view of the cylindrical ozone generating device (ozonizer) comprising safe (grounded) net electrode 1, dielectric spacer/film 2 that is placed between electrode 1 and base high voltage electrode 3. In disclosed configuration, high voltage electrode 3 is covered by said dielectric 2 and by lids 7 on the ends. Refrigerant 21, for example water-ice mix, can fill said cylinder, if it is necessary. Cooled ozonizer is more effective be placed in the container/room with dry air atmosphere. In this case ozone output is more (about 130 g/h per m2 in air). This ozonizer is very light, without refrigerant. This design especially convenient for high ozone productivity during several hours if cooled water fills said cylinder.

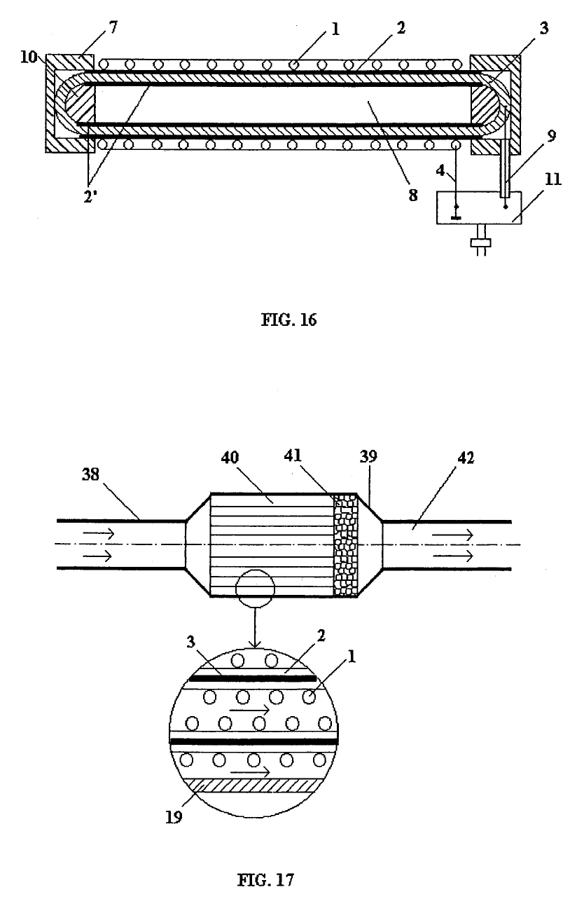

FIG. 16 shows a cross-section view of all sided safe (grounded) oval ozonizer that is refriged by running water, comprising safe (grounded) net electrode 1, said dielectric spacer/film 2 that is placed between electrode 1 and said base high voltage electrode 3. The net electrode 2 is grounded and the base electrode 3 is connected by insulated wire 9 with 3.5 kV, 60 Hz the power supply 11. The design of the ozonizer has a form similar to a flat tube flask. Opening 8, which is insulated inside by special spacers 10 and dielectric spacer/film 2′, which is identical to said dielectric spacer/film 2. The flanks of electrode 3 are insulated by plastic 7. Running water usually has a ground potential and is insulated from high-voltage electrode 3 by spacers 10 and spacer/film 2′. The low work voltage allows to use an electrical insulator 2″ that is 50-100 microns thick, which has breakdown voltage at least three times more than named work voltage and small heat resistance due to such small thickness. The cooling by running water is necessary for higher ozone output and ozonizer productivity during a long operational time.

FIG. 17 depict an air-ozone purifier for ventilation systems and like, in which entering air goes along conventional ventilator tube 38, which is connected with air-ozone purifier 40, Then the cleaned air gas goes through the filter 41 and exits through junction 39 into prolonging of conventional ventilation tube 42. Air-ozone purifier 40 comprises plurality of two sided generating elements, placed coaxial to each other forming a narrow gaps between ozone generating cylindrical elements, comprising the net electrode 1, base electrode 3 and dielectric spacer/film 2 between them. All said cylindrical generating elements are placed into cylindrical metal housing 19, which is grounded. The base cylindrical electrodes are connected between each other and connected with high voltage end of power supply. Net electrodes can be grounded. The air flow resistance of said narrow gaps is more than in conventional tubes 38 & 42 have and so diameter of air-ozone purifier housing 40 is more than diameter of said ventilator tubes. The electrical wind from corona surfaces into said gap increase treatment activity. Disclosed method provides corona and high ozone concentration in said gaps (reacting zone). The electrical field is practically absent in said reacting zone. But for some applications said field is needed. In such cases the net electrode is placed only on external cylinder surface. The cylindrical design has no limit for a cross section of reacting zones, because the number of said coaxial cylinders can be increased theoretically without limit. Depicted purifier is safe for ozone poison due to ozone/particles filter 41, which is filled by metal-oxides or other material granules for ozone conversion to oxygen. Said filter collects any dust too. This purifier can be used for every hospital, office, house, etc. and is convenient for improving not only ventilator systems. It can also be used in combustion process, increasing cetane number and lowering the compression ratio for Diesel engines. Moreover, said design can be used for cleaning the industrial chimney gas too. The ignition system of car/track can conveniently used as a power supply for discovered ozone purifier, having work voltage up to 3.6 kV.

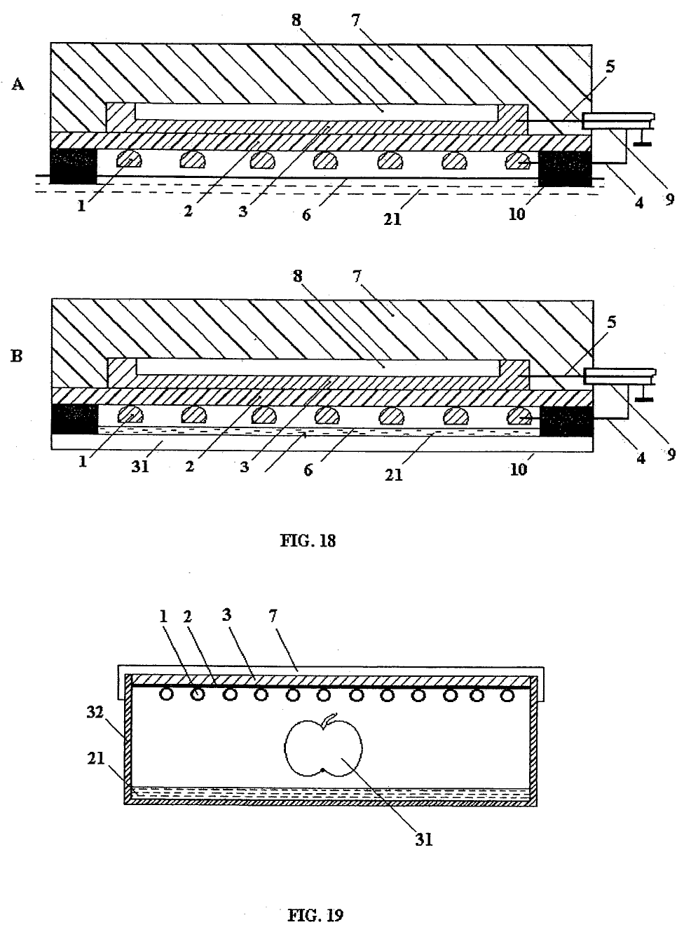

FIG. 18 is a cross-section view of the device for demolition (cool combustion) of organic (oil) film on the water surface (A) by ozone and for ozone saturation of liquid (for example, water) layer that moves above substrate, removing photoresist and other organic films from substrate (B).

The device has net electrode 1, dielectric spacer 2 and high voltage base electrode 3 which is covered by insulator 7. Said insulator has a cavity 8 for a cooling means. The wire 5 connects base electrode 3 with high voltage end of the power supply going through said insulator 7 and further going as cable 9. Said net electrode has a contact with the ground and so the contact with water is not dangerous. Two bars 10 allow the device to be floating (FIG. 18A) on the water 21 and determine the gap between said film 6 and corona surface. Due to OD method high concentration ozone fills this gap to provide ozone cool combustion of the organic film on the water or ozone dissolution in water. To compare with bubbles apparatus, disclosed ozonizer is more effective in practice because the porous plate for bubbles making and compressor are not needed. Thus the device disclosed in this patent is simpler and more cost effective.

The same design can be used for making high ozone concentration above water layer 2, which moves slowly on the substrate 31 (FIG. 18B). In this case the gap between net electrode 1 and said water layer 21 may be so small that corona is in direct contact with water surface 6. Using the Direct Corona Contact method for water surface is very effective for ozone dissolution in water due to electrical vortex wind contacting water surface 6. Ozonized water or any other liquid layer is moving along a substrate, as it is shown by arrow on FIG. 18B, to remove photoresist and other organic impurities from substrates surfaces 31.

Discovered design utilizing OD and/or DCC method make cleaning process simpler and cheaper. That is important and useful for applications in microelectronic, optics, and other industries, where cleaning processes are crucial.

FIG. 19 is a cross-section view of the device for ozone saturation of any thin layer of liquid or any subject in ozone containing gas by using ozone dispersion (OD) method. Liquid 21 is poured into a dielectric (plastic) vessel 32 having an insulator lid 7, The generating element comprising net electrode 1, base electrode 2 and dielectric spacer/film between them 3, is placed on the lid 7 facing liquid 21. The liquid 21 or treated subject 31 is kept for a necessary exposure time (several minutes) in a high ozone concentration atmosphere. The problem of keeping the saturated liquid (for example olive oil) for a long time is solved by keeping treated product in the simple box 31, together with ozone generating plate inside. The treatment process (liquid saturation or object sterilization) is conducted at the beginning and at the end of storing period. The conversion of the dissolved ozone into dissolved oxygen makes a supersaturated oxygen solution, because the solvent action for ozone gas in water is about ten times stronger than for oxygen gas and so ozone concentration in water can be more then limited oxygen concentration. For the saturated oil storage cooling means are usually used. Said means are not necessary if the device described here is used, because ozone solution can be made any time, minutes before usage. Food products 31 can be sterilized and stored for along time even moist. Storing moist meat products, vegetables and others is a problem, which often can not be solved by refrigeration because the appearance of ice crystals is not desired.

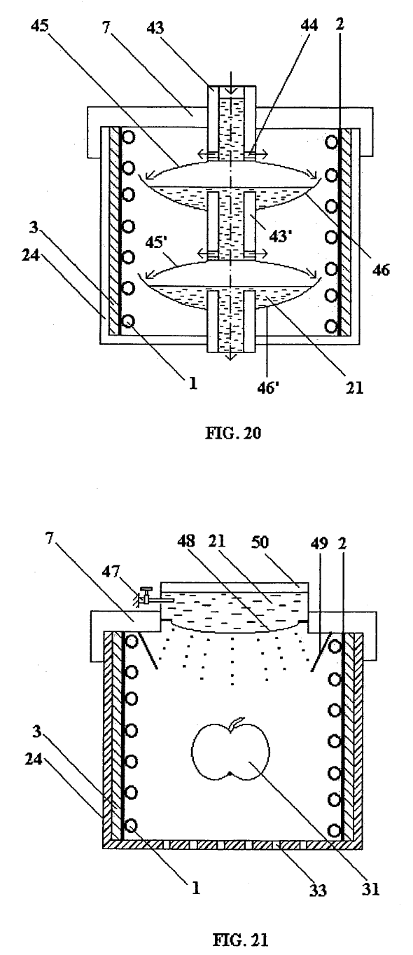

FIG. 20 depicts ozone device for disinfecting and saturation of viscosity liquids, which are used in medicine, food, chemical industry and especially in oil manufacturing. Liquid-film reactors are widely used and methods for calculation of appropriate apparatus are developed. According to this technology a thin liquid film/layer is formed on an inclined surface by a special shape of slits and effectively react with necessary gas reagent. In our case, the active reagent is a high concentrated ozone atmosphere. A treated liquid goes inside the vessel through the lid 7. The film/layer of liquid is disinfected and saturated by ozone in the film flowing process. A thin layer of liquid flows slowly, making ozone dissolution more effective. The design, provided by present patent, has an ozone generating cylindrical element having net electrode 1, base electrode 3 and dielectric spacer/film 2 between the said electrodes. The generating element is placed on the inner surface of a cylindrical vessel 24. Said vessel is made of an insulator to make the device safe. High voltage base electrode 3 is interposed between dielectric film 2 and a dielectric vessel wall and is fully insulated. The liquid to be treated enters through in the tube 43 that has several slits on the bottom to form liquid film/layer on the inclined surface of inverted dish 45. Said inclination is determined by the necessary contact time of the liquid film with ozone in order to destroy hundred percent of targeted bacteria or chemicals, or to saturate the liquid by ozone of necessary concentration. Then the liquid flows in the tube 43′ of the next cascade and then forms the film/layer on the inverted dish 45′of the next cascade. The number of cascades depends on the necessary productivity of the device. To increase the productivity it is necessary to increase the common velocity of the liquid current. As a result the time of ozone-liquid contact on one cascade decreases. Increasing the number of cascades increase the common contact time. It is possible to calculate the design for necessary productivity by using published data about said film technology.

Discovered ozone device uses OD method for treating said liquids and is novel. The film technology for ozone treatment is novel too. Discovered apparatus is simple and cost effective. For correct comparisons with the existing methods of viscosity liquid ozone treatment, it is necessary to account apparatus properties, including productivity, efficiency, power consumption, installation and service, cost, weight, dimensions, work life, etc.

FIG. 21 depicts ozone apparatus for water disinfecting and saturation by ozone, which uses a water shower in ozone containing air. The ozone saturated water drops can be used for decontamination and removing chemical compounds from objects such as medical instruments, food products, military objects (see patent to Ciambrone et al.), clothes, etc. Said apparatus comprise the inlet/water-tap 47 for filling the shower tank 48 with water and keeping necessary water pressure due to tight placed lid 50. Tank has small holes to create a water shower 48, which has a common design with dielectric led 7. A treating chamber 24 is made of dielectric and has ozone generating element inside. Ozone generating element has a grounded net electrode 1, a dielectric spacer/film 2, and high voltage main electrode 3, which is insulated from all sides. Cylindrical screens 49 are mounted on the lid 7 using to prevent corona discharge from direct contact with water drops. But water drops contact with the grounded net is not dangerous, it can only decrease ozone productivity. Water purification takes place due to ozone dissolution into the water drops, which go through ozone containing air/oxygen gas. Object 31 is placed under the ozonized shower to be disinfected. The holes 33 in the flour of said treating chamber 24 are used as an outlet for water. The net electrode and generating surface are absolutely safe.

Disclosed design can have a rectangular shape that is more convenient than cylindrical shape for placing flat ozone generating plates inside. The size of the drops is critical for its saturation with ozone, because contact surface of the drop increases relative to volume for smaller drops. The conveyer means can be executed along the length of said rectangular chamber.

The disclosed shower method and apparatus is much simpler, lighter, smaller and cheaper in comparison with the methods and apparatus that are discovered in the US patents to Cooper at al, to Burris et al and to Ciambrone et al. because ozone is not injected or bubbled in the water. Water shower apparatus is simple and inexpensive.

The logic of present patent is in corona/ozone generating element optimization that lead to some new physical effects that serve as basis for new application methods, which lead to the new apparatus for many applications. Although the foregoing invention has been described in some detail by way of illustration and example for purposes of clarity of understanding, it is obvious that certain changes and modifications may be practiced within the scope of the appended claims.

We claim:

1. A method for making ozone-generating element that creates corona surface discharge that produces ozone gas, comprising of the following steps:
applying a dielectric plate/film spacer, that has a specific capacity C′ equal to or more than 200 nanofarad per square meter;
applying a metal sheet, having a flat or cylindrical form—base electrode;
applying a net electrode from wire net or perforated metal or turned wire, said net electrode, having an open area not less than 70%, and diameter of holes or interval between near wire turns no more than 0.7V/P millimeters, if working voltage V in kilovolts, gas pressure P in atmospheres, and radius of wire or corresponding curvature of an edge of openings in perforated metal not less than 1.6 times of a thickness of the dielectric spacer;
placing said dielectric spacer between said base electrode and said net electrode;
connecting AC 0.7-3.6 kV power supply with said net electrode and said base electrode;
switching on said power supplier;
producing corona and ozone gas at the start voltage Vst=0.7+60/C′, kV, if C′ in nF/m² and
producing high output of ozone gas in grams per hour from one square meter of generating surface that grows intensively at the work voltage equal or more than 3Vₛₜ and
making surface corona discharge safe for touching by hand, if said net electrode is grounded and said base electrode is under work voltage up to 4 kV.

2. A method according to claim 1, wherein applied said net electrode has flat outside surface forming well-defined edges around net holes or slots formed by neighboring wire turns and whose thickness is not more than 20% of the diameter of the said holes or slots.

3. A method for application of the ozone generating element according to claim 1, comprising the steps:
placing the ozone generating element in a closed container/room with or without object to be treated;
switching on the power supply for ozone production;
keeping necessary ozone concentration that can be varied by work voltage, inside said container/room due to natural process of ozone dispersion from open corona surface;
switching off said power supply after necessary exposition;
applying ozone-oxygen converting means, for example UV lamps or water spray, to decrease ozone concentration to the permissible level in the time less than the time of ozone-oxygen natural conversion, if necessary;
opening said container/room, if it is necessary.

4. A method for Direct Corona Contact treatment by the ozone generating element as clamed in claim 2, comprising the steps:
grounding said net electrode to be safe,
placing said ozone generating element on the object to be treated in Direct Contact with said net electrode or having a small gap between the net electrode and treating surface so that the gap is filled by burning corona;
turning on a power supply to produce said corona surface discharge;
keeping burning corona in contact with said object during necessary time;
turning off said power supply after necessary exposition;
turning on a fan to flow the surrounding air along the ozone generating element on the treating surface;
removing said generating element from the treated surface of object.

5. A device for treating of an infected surface or a polymer surface by corona in various gases (nitrogen for example) using said Direct Corona Contact method according to claim 2 comprising:
said high voltage base electrode, having a special slot for cooling means;
cooling means, such as Peltier plates, for example;
said dielectric spacer/film, placed tightly on the base electrode;
said net electrode, placed on the said dielectric spacer/film;
an insulating lid to insulate said base electrode;
a periphery strip-spacer for making a closed volume formed by said periphery strip-spacer, said dielectric spacer/film and treatment surface, having series a small holes for filling said closed volume by different gases to make treating corona in necessary gas;
means to open and close holes in said periphery strip-spacer;
a conductor that dependably grounds net electrode;
an insulated conductor to connect said base electrode with power supply;
a power supply up to 3.6 kV AC, having a grounded end.

6. A device for disinfection/decontamination by using direct corona contact method according to claim 2, wherein both sides of a thin subjects such as letters, papers, banknote, etc. are treated in direct contact with corona, comprising:
two plain said ozone generating elements having fully insulated said base electrode and grounded said net electrodes that are placed face to face;
two strips-separators between said net electrodes limiting necessary gap between the net electrodes for placing said thin subject in this gap;
a press means to press the subject between said corona surfaces;
a power supply having a grounded end, that is connected with said net electrodes and high voltage insulated connector to connect said base electrodes;
a plastic lids having a plane slot for placing a cooling means for cooling said base electrodes, if it is necessary to cool;
a transport means to conveyor said subjects through said gap.

7. A device for disinfecting/deodorizing a toilet comprising:
a flat generating element that is made according the method clamed 2, which is placed on the inner side of toilet seat facing down to a bowl and having grounded the net electrode;
a relay to produce a command signal for switching said generating element on and off to provide necessary ozone exposure at necessary times;
a locker to lock or unlock said toilet seat cover;
a timer to establish necessary exposure for treatment of the bowl and deodorization of the air in a bowl;
a power supply with grounded end, which connects with said generating element having a grounded net electrode.

8. A device for ozone disinfection of subjects inside laundry washing/drying machines, according to method as claimed in claim 3 comprising:
said flat and thin generating element fitting tightly on a lid of said washing/drying machines having a safe generating surface facing inside said machine to create ozone atmosphere inside the drying machine or to obtain ozonized liquor inside the washing machines;
said base electrode that is insulated from all sides and having insulated high voltage contact with a power supply;
said net electrode that is grounded;
a controlled locker on the said lid;
a relay to produce commands to said lock and to the power supply to prevent the opening of the lid, when ozone gas is inside, and to provide the necessary treatment time;
a timer, connected to said relay to control the time of ozone generation and machine working time;
a controlled switch to switch on/off the ozone generating element and said machine.

9. A disinfecting device, according to claim 3, for treatment of objects inside a closed container in humid or dry atmosphere, comprising:
a metal container;
articles to be disinfected inside said container;
a metal lid on the said container;
said flat ozone generating element, placed on the inside surface of said lid and having said net electrode facing inside said container;
a high voltage net electrode that is connected with a power supply by insulated connector;
a detachable metal tank containing a water for making a humid atmosphere in said container, if necessary;
a heater to rise the temperature of the water to intensify the evaporation process and regulate the humidity;
a detachable metal tank containing a dryer material (silica gel, for example) to provide a dry atmosphere in said container;
a flash lamp (for example, Xenon lamp) or water spray to convert ozone into oxygen in a short time.

10. A device for ozone disinfection according to the method as claimed in claim 3 for mail, food, medical and other objects that are placed on the moving conveyer comprising:
a moving conveyer line;
objects placed on the conveyer line to be treated;
a closed room, filled by ozone-air mixture, placed on a segment of said conveyor;
said ozone generating element with said net electrode facing the conveyer line and placed inside said room;
an entrance and exit buffer volumes to prevent ozone penetration of the surrounding air;
flexible screens on the entrance buffer volume and two on the exit buffer volume are placed to close the entrance and the exit to prevent ozone penetrating in the surrounding air and at the same time to allow treated objects to pass through.
a fan to provide a gas current across to the movement direction of the conveyer, placed in the exit buffer volume between two said screens to form a gas shutter to prevent ozone escaping from said room into the surrounding air;
an ozone-oxygen converter means placed along the way of the said transverse gas current and consisting of numerous small gaps between plates made of material having a big ozone converting/destroying ability to prevent ozone penetrating in the surrounding air (such as); (do we really need that) a power supply safely connected with said generating element.

11. A device for ozone disinfection and storage of poisonous objects in a closed bag/container that can be safely and cost effectively thrown out in the trash with the container and the disinfected material, according to the method as claimed in claim 3, comprising:
a bag/container;
said ozone generating element that is a lid of said bag/container with base electrode grounded;
a separating dielectric plate having a numerous openings that is placed between treated objects and the net electrode of said ozone generating element;
a support-plate made of a heat conductive material having holes is used to support the objects to be disinfected;
a detachable cooling or drying box that is placed underneath the support-plate for cooling or drying of said object,
a coolant (for example, water-ice mixture or Peltier plate) or a dryer (for example silica gel) that is placed in the said box depending the object to be disinfected;
a power supply connecting with said generating element safely.

12. An ozone generating device for ozone-air deodorization according to the method as claimed in claim 3 executing in the form of picture, comprising:
net electrode that is grounded and made of a perforated metal plate, having slots in a form of graphic picture, which will glow blue in a dark;
said dielectric spacer/film placed tightly on the said base electrode;
said base electrode connecting with high voltage end of power supply;
an insulation that covers said base electrode,
a power supply executed as a plug and connected with said ozone generating device by cable;

13 A device for ozone disinfection of the whole human/animal body according to the method as claimed in claim 3 comprising:
thin metal box that is insulated and is the high voltage base electrode;
an insulator that covers a base electrode outside to make it completely safe;
a generating element with grounded safe corona facing inside of the said box and placed on the inner wall of said box;
an upper lid that made of insulator and has a hole for patient's neck;
a special “scarf” to prevent the patient from ozone odor;
a fan for blowing ambient air in a patient face to prevent the patient from ozone odor;
a power supply with one end grounded and other high voltage end is connected by insulated connector with said base electrode.

14. A device for ozone purification of ventilated air according to the method as claimed in claim 3, comprising:
a conventional ventilator tube;
an enter junction that is placed between said ventilator tube and larger in diameter air-ozone purifier;
an air-ozone purifier comprising of a several high voltage cylindrical ozone generating elements with different diameters and placed coaxial one into another forming a number of thin cylindrical gaps that the air flows through;
a filter placed near the exit junction of said air-ozone purifier;
an exit junction placed between said air-ozone purifier and the continuation of the conventional ventilator tube;
a power supply having one end grounded that is connected with said base electrode and other high voltage end that is connected with said net electrode.

15 a device for demolition of oil films on the water, or making high ozone concentration in a thin water layer that covers substrates to remove photoresist or other organic impurities according to the method as claimed in claim 5, characterized in that the said net electrode is supported by strip spacers and floats on water surface, to form necessary air gap between the net electrode and treated water surface or organic film on the water surface. In a case of removing photoresist from substrate surface, said strip spacers are placed on the substrate to allow for a necessary gap between grounded corona surface and thin water layer that moves slowly along substrate surface to saturate said layer by ozone.

16. A device for ozone treating of objects, including ozone saturation of liquids, or sterilization of food, or medical articles, according to the method as claimed in claim 3, comprising:
a vessel made of dielectric having no more than about 10 centimeters in depth;
a lid made of dielectric to cover said vessel tightly;
said ozone generating element that is placed on the lid with net electrode facing inside of the vessel;
a power supply with grounded end, connected with said ozone generating element;
a thin layer of the liquid to be saturated (for example olive oil) or objects to be sterilized inside said vessel;
means to control necessary exposure, depending on the object/subject.

17. A device for ozone treatment of viscosity liquids that are used in medical, food and oil industries, by using film-reagent technology, according to the method as claimed in claim 3, comprising:
a cylindrical chamber made of a dielectric;
said ozone generating element of a cylindrical form, that is placed inside said cylindrical chamber on its wall;
a lid on the top of said chamber;
a guide inlet tube passing through said led and having slits on it's bottom to form liquid film/layer on the inclined surface;
an inverted dish having inclined surface to allow for slow movement of said film on it, in ozone atmosphere;
a storage dish that is filled by the liquid to be treated, which flows as a film from said inclined surface in that dish;
a level tube that determine the level of said liquid in the storage dish that has said slits in the bottom to form liquid film/layer on the inclined surface of the second inverted dish;
second inverted dish;
a second storage dish that is filled by treated liquid up to the limit that is determined by the height of the output level tube;
an output level tube that determines the level of said liquid in the second storage and provides an exit for the liquid to the bottom of said chamber.

18 A device for ozone disinfection, decolorization and deodorization of water shower by ozone atmosphere that (water) can be used to treat various objects, according to the method as claimed in claim 3, comprising:
a chamber for ozone treating of cylindrical or rectangular shape;
said ozone generating elements having a grounded net electrode, placed on the chamber wall (or walls if a chamber has a rectangular form);
an upper lid of said chamber;
a tank placed in the opening of said lid to form shower ceiling of said chamber;
an inlet/water-tap that fills said tank of water and keeps necessary water pressure in the tank;
a screen/screens to prevent corona discharge from direct contact with water drops, which can reduce ozone generation output, but can not make a short-circuit of the ozone generating element;
a subject to be treated by ozonized water and by ozone atmosphere simultaneously;
a bottom of said chamber, having outlets for water;
a power supply connecting with said ozone generating element.
